(19) United States
(12) Patent Application Publication
(10) Pub. No.: US 2024/0161933 A1
(43) Pub. Date: May 16, 2024

(54) PREDICTION OF POST-OPERATIVE PAIN USING HOSVD

(71) Applicant: University of Florida Research Foundation, Incorporated, Gainesville, FL (US)

(72) Inventors: Raheleh BAHARLOO, Gainesville, FL (US); Patrick J. TIGHE, Gainesville, FL (US); Parisa RASHIDI, Gainesville, FL (US); Jose C. PRINCIPE, Gainesville, FL (US); Arash ANDALIB, Gainesville, FL (US)

(21) Appl. No.: 18/552,486

(22) PCT Filed: Jun. 7, 2022
(86) PCT No.: PCT/US2022/032427

§ 371 Date: Sep. 26, 2023

Related U.S. Application Data
(60) Provisional application No. 63/202,374, filed on Jun. 8, 2021.

(51) Int. Cl. G16H 50/30; G06N 5/022

(52) U.S. Cl. CPC G16H 50/30; G06N 5/022

(57) ABSTRACT

Various embodiments of the present disclosure provide systems and methods for prediction of a risk for mild or severe persistent post-operative pain (POP) for an individual of interest. A risk prediction may be determined based at least in part on a cohort predictive model. The cohort predictive model is associated with a surgical type cohort and initialized with historical multivariate intra-operative vital sign data associated with binary classifications of mild or severe persistent post-operative pain. Using complex higher-order singular value decomposition, phase information for the historical multivariate intra-operative vital sign data is determined. A relationship between phase information and mild or severe persistent POP is then determined using discriminant analysis. Subsequently, phase information for multivariate intra operative vital sign data for an individual of interest is provided to a cohort predictive model, which uses the determined relationship to classify the individual of interest. The risk prediction then comprises the classification.

### CROSS-REFERENCE TO RELATED APPLICATION

The present application claims benefit under 35 USC 119(e) of U.S. Application Ser. No. 63/202,374, filed Jun. 8, 2021, which is incorporated herein by reference in its entirety.

### GOVERNMENT SUPPORT

This invention was made with government support under grant number R01 GM114290, awarded by the National Institutes of Health. The government has certain rights in the invention.

### TECHNOLOGICAL FIELD

Embodiments of the present disclosure generally relate to systems and methods for post-operative pain (POP) risk prediction based on biological and biomedical measurements.

### BACKGROUND

Long-term pain conditions after surgery and an individual's response to pain relief medications are not yet fully understood. More than 100 million patients undergo surgery each year in the US. More than 60 percent of these patients suffer from acute post-operative pain. Pain resolution after surgery is highly variable: one-third of patients experience stable or even increasing pain on each day after surgery for at least seven days after the surgery.

Persistent pain after acute post-operative pain (POP) is experienced by 10-50% of individuals after common surgical procedures like cardiac, thoracic, spine, or orthopedic surgeries. Although even mild levels of persistent post-operative pain (POP) are associated with decreased physical and social activities, 2-10% of patients experiencing this type of pain may develop severe levels of pain, hence delaying recovery and their return to normal daily function. Furthermore, persistent POP leads to increased direct medical costs through additional resource use. Prediction, identification, and assessment of persistent POP is a critical and unrecognized clinical problem. Consequently, recognition of patients at risk of developing this type of pain has remained inadequate.

POP is assumed to stem from various interacting factors including, but not limited to, biological, psychological, and social factors. For example, psychological factors (depression, psychological vulnerability, stress, and catastrophizing) may be risk factors for development of persistent POP. As another example, the female gender may be a risk factor for developing persistent POP. More significantly, the severity of acute POP, and especially movement-evoked pain, is a major risk factor significantly associated with persistent POP. In such cases, neuroplastic changes in the central nervous system resulting from high intensities of acute POP may be a cause of the development of persistent POP.

### BRIEF SUMMARY

In general, embodiments of the present disclosure provide methods, apparatuses, systems, computing devices, computing entities, and/or the like for predicting a risk of persistent post-operative pain (POP) for an individual and performing one or more risk prediction-based actions. In various embodiments, multivariate intra-operative vital sign data for a cohort of individuals may be collected and processed. Each individual may be associated with a binary classification indicating whether the individual experienced mild or severe persistent POP. Processing the multivariate intra-operative vital sign data may involve performing complex higher-order singular value decomposition (HOSVD) techniques to generate a plurality of dimensional representations. For example, the multivariate intra-operative vital sign data may be structured as a three-dimensional tensor (e.g., with one dimension representing different vital sign variates, another dimension representing intra-operative time, and yet another dimension representing different individuals), and processing the multivariate intra-operative vital sign data may result in a plurality of first dimension mode data objects, a plurality of second dimension mode data objects, and a plurality of third dimension mode data objects.

In various embodiments, a cohort predictive model for the cohort may be generated based at least in part on the processing of the multivariate intra-operative vital sign data for the cohort of individuals. The cohort predictive model may be initialized with the multivariate intra-operative vital sign data for the cohort of individuals to determine a relationship between phase information of multivariate intra-operative vital sign data. In various embodiments, a risk prediction for persistent POP for an individual of interest may be generated and provided based at least in part on providing multivariate intra-operative vital sign data for the individual of interest to a cohort predictive model associated with a cohort to which the individual of interest belongs. The multivariate intra-operative vital sign data for the individual of interest may be processed (e.g., via Hilbert transform techniques) to determine phase information, and the cohort predictive model may determine a binary classification for the individual of interest of mild or severe persistent POP using the multivariate intra-operative vital sign data and/or phase information of the multivariate intra-operative vital sign data. Thus, in various embodiments, the risk prediction for the individual of interest comprises the binary classification of mild or severe persistent POP. In various embodiments, various risk prediction-based actions may then be performed for the individual.

In some embodiments, a computer-implemented method for predicting a risk of persistent post-operative pain for an individual includes, in part, receiving, by a processor, a prediction input data object comprising multivariate intra-operative vital sign data of the individual; processing the multivariate intra-operative vital sign data of the individual; providing at least the processed multivariate intra-operative vital sign data to a cohort predictive model associated with a cohort of the individual, wherein the cohort predictive model is initialized with historical data objects associated with a post-operative timepoint; generating a risk prediction data object comprising a classification of phase information determined based at least in part on the cohort predictive model, wherein the risk prediction data object is associated with the post-operative timepoint; and performing one or more risk prediction-based actions for the individual.

In some embodiments, processing the multivariate intra-operative vital sign data comprises complexifying the multivariate intra-operative vital sign data of the individual. In some embodiments, complexing the multivariate intra-operative vital sign data of the individual comprises augmenting the multivariate intra-operative vital sign data with their Hilbert transform.

In some embodiments, providing at least the processed multivariate intra-operative vital sign data to a cohort predictive model comprises projecting the processed multivariate intra-operative vital sign data onto a three-dimensional manifold of the cohort predictive model and determining phase information of the projection of the processed multivariate intra-operative vital sign data.

In some embodiments, the cohort predictive model is generated and initialized based at least in part by receiving a historical data object for each of a cohort comprising a plurality of individuals, each historical data object associated with a binary classification and comprising multivariate intra-operative vital sign data for a corresponding individual; processing the plurality of historical data objects to generate a plurality of first dimension mode data objects, a plurality of second dimension mode data objects, and a plurality of third dimension mode data objects; generating a cohort predictive model based at least in part on the plurality of first dimension mode data objects and the plurality of second dimension mode data objects, wherein the plurality of first dimension mode data objects and the plurality of second dimension mode data objects are processed to generate a three-dimensional manifold; and initializing the cohort predictive model with the plurality of historical data objects based at least in part on the plurality of third dimension mode data objects and each binary classification.

In some embodiments, the plurality of historical data objects is aggregated and processed together using complex higher-order singular value decomposition (HOSVD), and the three-dimensional manifold is generated based at least in part on ranks of components generated by the HOSVD. In some embodiment, the ranks of components may be determined using a rank feature method based at least in part on Fisher ranking techniques. In some embodiment, the top three ranked components are selected to form the three-dimensional manifold.

In some embodiments, each of the plurality of first dimension mode data objects comprises a weight for each of one or more vital sign variate types; each of the plurality of second dimension mode data objects comprises a weight for each of a plurality of intra-operative timepoints; and each of the plurality of third dimension mode data objects comprises a weight for each of the plurality of individuals.

In some other embodiments, the plurality of first dimension mode data objects comprises eigenvectors of a first correntropy matrix, wherein the first correntropy matrix is generated based at least in part on the plurality of historical data objects; the plurality of second dimension mode data objects comprises eigenvectors of a second correntropy matrix, wherein the second correntropy matrix is generated based at least in part on the plurality of historical data objects; and the plurality of third dimension mode data objects comprises eigenvectors of a third correntropy matrix, wherein the third correntropy matrix is generated based at least in part on the plurality of historical data objects. In some embodiments, the first correntropy matrix is generated by applying a first cross-correntropy function to a first moment matrix, wherein the first moment matrix is generated based at least in part on a first mode matrix unfolding of a third-order tensor; the second correntropy matrix is generated by applying a second cross-correntropy function to a second moment matrix, wherein the second moment matrix is generated based at least in part on a second mode matrix unfolding of the third-order tensor; and the third correntropy matrix is generated by applying a third cross-correntropy function to a third moment matrix, wherein the third moment matrix is generated based at least in part on a third mode matrix unfolding of the third-order tensor, wherein the third-order tensor represents the plurality of historical data objects. In some embodiments, each of the first, second, and third cross-correntropy functions is based on a Gaussian function.

In some embodiments, initializing the cohort predictive model comprises determining a relationship between phase information of the projection of the plurality of historical data objects onto the three-dimensional manifold and a binary classification.

In some embodiments, the one or more risk prediction-based actions for the individual comprises displaying the risk prediction data object with a three-dimensional manifold, wherein the three-dimensional manifold is generated based at least in part on the historical data objects.

In some embodiments, an apparatus for predicting a risk of persistent post-operative pain for an individual comprises at least one processor and at least one non-transitory memory including program code. The at least one non-transitory memory and the program code are configured to, with the at least one processor, cause the apparatus to at least receive a prediction input data object comprising multivariate intra-operative vital sign data of the individual; process the multivariate intra-operative vital sign data of the individual; provide at least the processed multivariate intra-operative vital sign data to a cohort predictive model associated with a cohort of the individual, wherein the cohort predictive model is initialized with historical data objects associated with a post-operative timepoint; generate a risk prediction data object comprising a classification of phase information determined based at least in part on the cohort predictive model, wherein the risk prediction data object is associated with the post-operative timepoint; and perform one or more risk prediction-based actions for the individual.

In some embodiments, configuring the at least one non-transitory memory and the program code to, with the at least one processor, cause the apparatus to process the multivariate intra-operative vital sign data comprises configuring the at least one non-transitory memory and the program code to, with the at least one processor, cause the apparatus to complexify the multivariate intra-operative vital sign data of the individual. In some embodiments, configuring the at least one non-transitory memory and the program code to, with the at least one processor, cause the apparatus to complexify the multivariate intra-operative vital sign data of the individual comprises configuring the at least one non-transitory memory and the program code to, with the at least one processor, cause the apparatus to augment the multivariate intra-operative vital sign data with their Hilbert transform.

In some embodiments, configuring the at least one non-transitory memory and the program code to, with the at least one processor, cause the apparatus to provide at least the processed multivariate intra-operative vital sign data to a cohort predictive model comprises configuring the at least one non-transitory memory and the program code to, with the at least one processor, cause the apparatus to project the processed multivariate intra-operative vital sign data onto a three-dimensional manifold of the cohort predictive model and determine phase information of the projection of the processed multivariate intra-operative vital sign data.

In some embodiments, the cohort predictive model that the apparatus is configured to provide at least the processed multivariate intra-operative vital sign data to is generated and initialized based at least in part by receiving a historical data object for each of a cohort comprising a plurality of individuals, each historical data object associated with a binary classification and comprising multivariate intra-operative vital sign data for a corresponding individual; processing the plurality of historical data objects to generate a plurality of first dimension mode data objects, a plurality of second dimension mode data objects, and a plurality of third dimension mode data objects; generating a cohort predictive model based at least in part on the plurality of first dimension mode data objects and the plurality of second dimension mode data objects, wherein the plurality of first dimension mode data objects and the plurality of second dimension mode data objects are processed to generate a three-dimensional manifold; and initializing the cohort predictive model with the plurality of historical data objects based at least in part on the plurality of third dimension mode data objects and each binary classification.

In some embodiments, each of the plurality of first dimension mode data objects comprises a weight for each of one or more vital sign variate types; each of the plurality of second dimension mode data objects comprises a weight for each of a plurality of intra-operative timepoints; and each of the plurality of third dimension mode data objects comprises a weight for each of the plurality of individuals.

In some other embodiments, the plurality of first dimension mode data objects comprises eigenvectors of a first correntropy matrix, wherein the first correntropy matrix is generated based at least in part on the plurality of historical data objects; the plurality of second dimension mode data objects comprises eigenvectors of a second correntropy matrix, wherein the second correntropy matrix is generated based at least in part on the plurality of historical data objects; and the plurality of third dimension mode data objects comprises eigenvectors of a third correntropy matrix, wherein the third correntropy matrix is generated based at least in part on the plurality of historical data objects. In some embodiments, the first correntropy matrix is generated by applying a first cross-correntropy function to a first moment matrix, wherein the first moment matrix is generated based at least in part on a first mode matrix unfolding of a third-order tensor; the second correntropy matrix is generated by applying a second cross-correntropy function to a second moment matrix, wherein the second moment matrix is generated based at least in part on a second mode matrix unfolding of the third-order tensor; and the third correntropy matrix is generated by applying a third cross-correntropy function to a third moment matrix, wherein the third moment matrix is generated based at least in part on a third mode matrix unfolding of the third-order tensor, wherein the third-order tensor represents the plurality of historical data objects. In some embodiments, each of the first, second, and third cross-correntropy functions is based on a Gaussian function.

In some embodiments, to generate and initialize the cohort predictive model, the plurality of historical data objects is aggregated and processed together using complex higher-order singular value decomposition (HOSVD), and the three-dimensional manifold is generated based at least in part on ranks of components generated by the HOSVD. In some embodiments, the ranks of components may be determined using a rank feature method based at least in part on Fisher ranking techniques. In some embodiments, the top three ranked components are selected to form the three-dimensional manifold. In some embodiments, initializing the cohort predictive model comprises determining a relationship between phase information of the projection of the plurality of historical data objects onto the three-dimensional manifold and a binary classification.

In some embodiments, configuring the at least one non-transitory memory and the program code to, with the at least one processor, cause the apparatus to perform the one or more risk prediction-based actions for the individual comprises configuring the at least one non-transitory memory and the program code to, with the at least one processor, cause the apparatus to display the risk prediction data object with a three-dimensional manifold, wherein the three-dimensional manifold is generated based at least in part on the historical data objects.

### BRIEF DESCRIPTION OF THE DRAWINGS

Having thus described embodiments of the present disclosure in general terms, reference will now be made to the accompanying drawings, which are not necessarily drawn to scale, and wherein:

FIG. 1 provides an exemplary overview of an example system architecture that may be used to practice various embodiments of the present disclosure;

FIG. 2 is a schematic of an example system computing entity in accordance with various embodiments of the present disclosure;

FIG. 3 is a schematic of an example client computing entity in accordance with various embodiments of the present disclosure;

FIG. 4 provides a block diagram of an example system computing entity in accordance with various embodiments of the present disclosure;

FIGS. 5A and 5B provide process flows of example operations for predicting a risk of post-operative pain in accordance with various embodiments of the present disclosure;

FIG. 6 illustrates portions of some example cohort predictive models, in accordance with some embodiments of the present disclosure;

FIG. 7 provides a diagram of an example process for predicting a risk of post-operative pain in accordance with various embodiments of the present disclosure;

FIG. 8 illustrates portions of some example cohort predictive models, in accordance with some other embodiments of the present disclosure;

FIGS. 9A and 9B show the first three temporal factors obtained using two different example sets of kernel width, in accordance with some embodiments of the present disclosure;

FIG. 10 shows the first three temporal factors obtained using an example optimal kernel width, in accordance with some embodiments of the present disclosure; and

FIG. 11 shows example changes of the value of Fisher scores in the top ten components extracted by applying an example complex HOSVD for different example sets of Kernel width, in accordance with some embodiments of the present disclosure.

### DETAILED DESCRIPTION

Various embodiments of the present disclosure now will be described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that the present disclosure will satisfy applicable legal requirements. The term “or” (also designated as “/”) is used herein in both the alternative and conjunctive sense, unless otherwise indicated. The terms “illustrative” and “exemplary” are used to be examples with no indication of quality level. Like numbers refer to like elements throughout.

### I. General Overview and Technical Advantages

Various embodiments of the present disclosure generate cohort predictive models for determining a relationship between phase information of multivariate intra-operative vital sign data and mild or severe persistent POP. Various embodiments further apply such determined relationships to predict whether an individual of interest may develop mild or severe persistent POP based at least in part on the phase information of multivariate intra-operative vital sign data for the individual of interest. By doing so, various embodiments advantageously consider each individual's unique systematic response to surgical injury in relation to development of persistent post-operative POP.

During surgery, as the autonomic nervous system continuously responds to various surgical stimuli, different vital sign variate types such as heart rate, blood pressure, and respiration can be used as indicators of individuals' systematic responses. During general anesthesia, when a sufficient dose of anesthetic agent is applied to prevent the response to skin incision, hemodynamic responses induced by surgical stress are not necessarily attenuated. The sympathetic nervous system inherently changes hemodynamic parameters such as local blood flow, blood pressure, and heart rate in response to noxious stimulation. Anesthetic agents do interfere with this system at different levels. Among hemodynamic parameters, heart rate may also include changes in parasympathetic discharge. Hence, monitoring and analyzing the time series of patients hemodynamic responses in relation to a variety of surgical stimuli and nociception imbalance under general anesthesia indirectly characterizes the behavior of the autonomic nervous system to nociceptive stimuli and provides a relationship with the development of persistent POP.

Various embodiments of the present disclosure employ complex HOSVD to explore dynamic correlations with lead/lag relations in intra-operative vital signs. In various embodiments, complex vital sign data is generated using Hilbert transform techniques. Multivariate-temporal structure of intra-operative vital signs is revealed by quantifying cross correlations of the data as a joint function of vital sign variate types and time. As such, various embodiments advantageously employ complex HOSVD to compress correlation structures into a rather few number of complex eigenvectors. The complex eigenvectors are employed as new bases to describe hemodynamic responses. After projection onto a subspace with the new bases, the complex correlations between each intra-operative time series and the eigenvectors are manifested in magnitudes and phases of the correlations. In various embodiments, the phases of the correlations are used to infer lead/lag relations in the original intra-operative time series.

In various embodiments, multivariate intra-operative vital sign data comprises intra-operative time series recorded for different vital sign variate types, such as heart rate, blood oxygen level, end-tidal CO2 levels, respiratory tidal volume, systolic blood pressure, diastolic blood pressure, isoflurane concentration, sevoflurane concentration, and/or the like. Various embodiments may use multivariate intra-operative vital sign data for a cohort of individuals for generating a cohort predictive model. In various embodiments, the multivariate intra-operative vital sign data for the cohort is organized in a three-dimensional tensor Aϵ [symbol] ^{I}^{₁}^{×I}^{₂}^{×I}^{₃}, where I₁ and I₂ represent the number of vital sign variate types and the number of intra-operative timepoints (e.g., periodic timepoints when vital sign data are collected) and I₃ is the number of individuals or patients in the cohort. In various embodiments, cohort predictive models may be generated for different cohorts determined based at least in part on surgical operation type, such as orthopedic, urology, colorectal, transplant, pancreatic/biliary, and thoracic surgeries. In various embodiments, the cohort predictive models may determine a difference in phase information between individuals of a cohort who developed mild persistent POP at 30 days after operation and individuals of a cohort who developed severe persistent POP at 30 days after operation. In various embodiments, the cohort predictive models may additionally or alternatively determine a difference in phase information between individuals who developed mild persistent POP at 90 days after operation and individuals who developed severe persistent POP at 90 days after operation.

Indeed, various embodiments of the present disclosure provide technical advantages and improvements to various other methods and systems for analyzing multivariate intra-operative vital sign data. For example, cross-spectral analysis is difficult to employ and less descriptive for irregularly occurring events and unknown dominant frequencies of dynamic interactions between coupled biological systems in hemodynamic regulation. Furthermore, various embodiments advantageously determine phase information related to propagating dynamics of hemodynamic responses, as opposed to standing dynamics. In general then, various embodiments of the present disclosure are uniquely and advantageously suited to accurately predict a risk of persistent POP for an individual of interest based at least in part on an analysis of the individual's inherent response to painful stimulus captured in the multivariate intra-operative vital sign data.

### II. Exemplary System Architectures

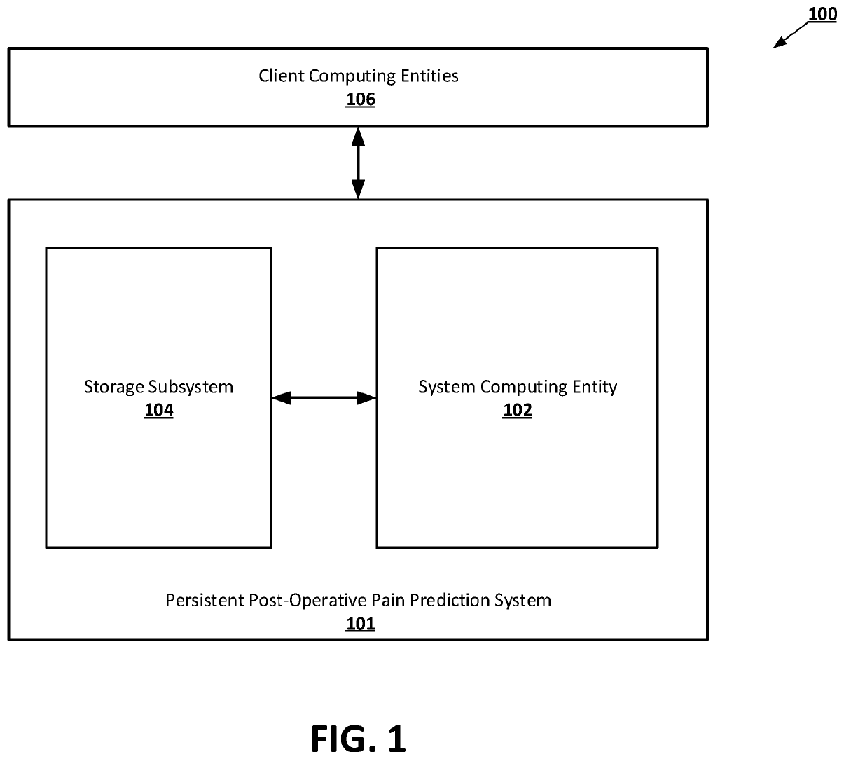

FIG. 1 is a schematic diagram of an example system architecture 100 for predicting a risk of persistent POP for an individual and performing one or more risk prediction-based actions. The system architecture 100 includes a persistent POP prediction system 101 configured to generate cohort predictive models, generate and provide risk prediction data objects for an individual of interest based at least in part on the cohort predictive models, perform one or more risk prediction-based actions, and/or the like. In various embodiments, the persistent POP prediction system 101 provides a risk prediction data object for an individual of interest based at least in part on receiving a prediction input data object from a client computing entity 106.

In some embodiments, the persistent POP prediction system 101 may communicate with at least one of the client computing entities 106 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like). In various embodiments, the persistent POP prediction system 101 comprises an application programming interface (API), receives a prediction input data object from a client computing entity 106 via an API call, and provides a risk prediction data object via an API response.

The persistent POP prediction system 101 may include a system computing entity 102 and a storage subsystem 104. The system computing entity 102 may be configured to generate cohort predictive models, receive prediction input data objects from one or more client computing entities 106, process a prediction input data object, and provide a risk prediction data object based at least in part on providing the prediction input data object to a cohort predictive model. In various embodiments, the system computing entity 102 is a cloud-based computing system and comprises one or more computing devices each configured to share and allocate computer processing resources and data.

The storage subsystem 104 may be configured to store data for predicting a risk of persistent POP for an individual and for performing one or more risk prediction-based actions. For example, cohort predictive models generated by the system computing entity 102 may be stored in the storage subsystem 104. The storage subsystem 104 may include one or more storage units, such as multiple distributed storage units that are connected through a computer network. Each storage unit in the storage subsystem 104 may store at least one of one or more data assets and/or one or more data about the computed properties of one or more data assets. Moreover, each storage unit in the storage subsystem 104 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### III. Exemplary Computing Entities

In general, the terms device, system, computing entity, entity, and/or similar words used herein interchangeably can refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

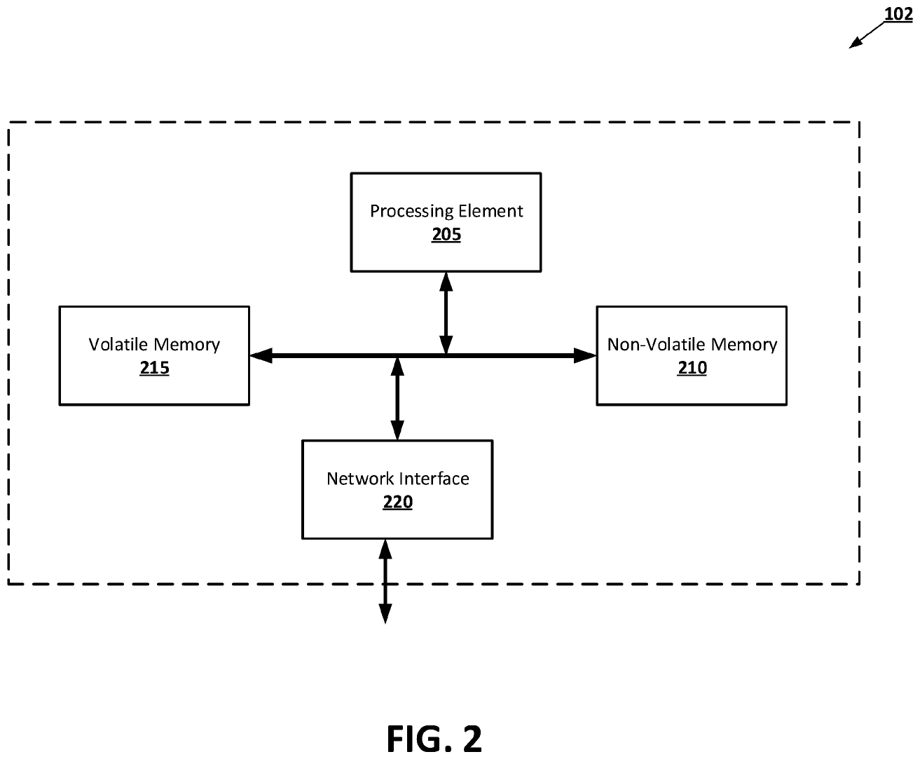

FIG. 2 provides an illustrative schematic representative of a system computing entity 102 that can be used in conjunction with embodiments of the present disclosure. For instance, the system computing entity 102 may be configured to and/or comprise means for generating cohort predictive models, generating and providing persistent POP risk prediction data objects, and performing one or more risk prediction-based actions. As shown in FIG. 2, in one embodiment, the system computing entity 102 may include, or be in communication with, one or more processing elements 205 (also referred to as processors, processing circuitry, and/or similar terms used herein interchangeably) that communicate with other elements within the system computing entity 102 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In one embodiment, the system computing entity 102 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media 210 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the system computing entity 102 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media 215 may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the system computing entity 102 with the assistance of the processing element 205 and operating system.

As indicated, in one embodiment, the system computing entity 102 may also include one or more network interfaces 220 for communicating with various computing entities (e.g., one or more client computing entities 106), such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the system computing entity 102 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the system computing entity 102 may include, or be in communication with, one or more input elements, such as a keyboard input, a mouse input, a touch screen/display input, motion input, movement input, audio input, pointing device input, joystick input, keypad input, and/or the like. The system computing entity 102 may also include, or be in communication with, one or more output elements (not shown), such as audio output, video output, screen/display output, motion output, movement output, and/or the like.

As will be appreciated, one or more of the components of the system computing entity 102 may be located remotely from other components, such as in a distributed system. Furthermore, one or more of the components may be aggregated and additional components performing functions described herein may be included in the system computing entity 102. Thus, the system computing entity 102 can be adapted to accommodate a variety of needs and circumstances.

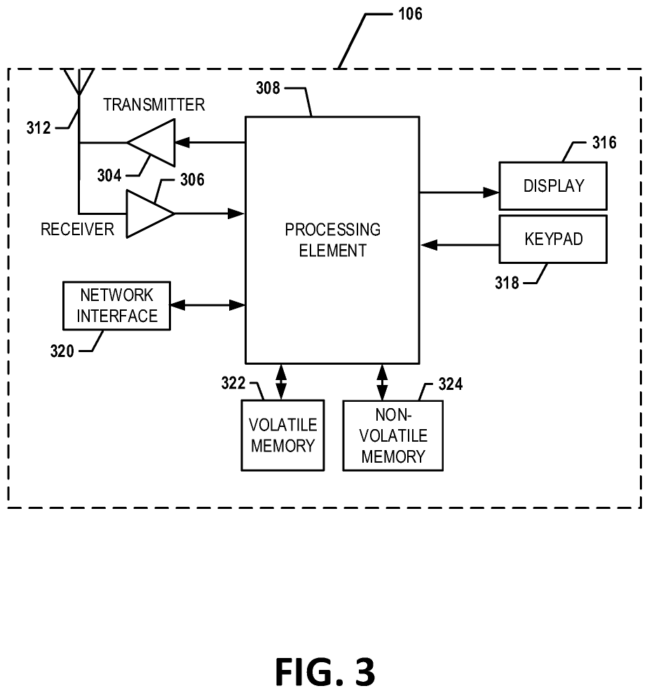

FIG. 3 provides a schematic of an example client computing entity 106 that may be used in conjunction with embodiments of the present disclosure. Client computing entities 106 can be operated by various parties, and the system architecture 100 may include one or more client computing entities 106. As shown in FIG. 3, the client computing entity 106 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 106 may be capable of operating with one or more air interface standards, communication protocols, modulation types, and access types. More particularly, the client computing entity 106 may operate in accordance with any of a number of wireless communication standards and protocols, such as those described above with regard to the system computing entity 102. In a particular embodiment, the client computing entity 106 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 106 may operate in accordance with multiple wired communication standards and protocols, such as those described above with regard to the system computing entity 102 via a network interface 320.

Via these communication standards and protocols, the client computing entity 106 can communicate with various other entities (e.g., system computing entities 102, storage subsystem 104) using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 106 can also download changes, add-ons, and updates, for instance, to its firmware, software (e.g., including executable instructions, applications, program modules), and operating system.

According to one embodiment, the client computing entity 106 may include location determining aspects, devices, modules, functionalities, and/or similar words used herein interchangeably. For example, the client computing entity 106 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 106 position in connection with a variety of other systems, including cellular towers, Wi-Fi access points, and/or the like. Similarly, the client computing entity 106 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 106 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 106 to interact with and/or cause display of information/data from the system computing entity 102, as described herein. The user input interface can comprise any of a number of devices or interfaces allowing the client computing entity 106 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 106 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 106 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 106. As indicated, this may include a user application that is resident on the entity or accessible through a browser or other user interface for communicating with the system computing entity 102, various other computing entities, and/or a storage subsystem 104.

In another embodiment, the client computing entity 106 may include one or more components or functionality that are the same or similar to those of the system computing entity 102, as described in greater detail above. As will be recognized, these architectures and descriptions are provided for exemplary purposes only and are not limiting to the various embodiments.

In various embodiments, the client computing entity 106 may be embodied as an artificial intelligence (AI) computing entity, such as an Amazon Echo, Amazon Echo Dot, Amazon Show, Google Home, and/or the like. Accordingly, the client computing entity 106 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### IV. Exemplary System Operations

### Model Generation Module

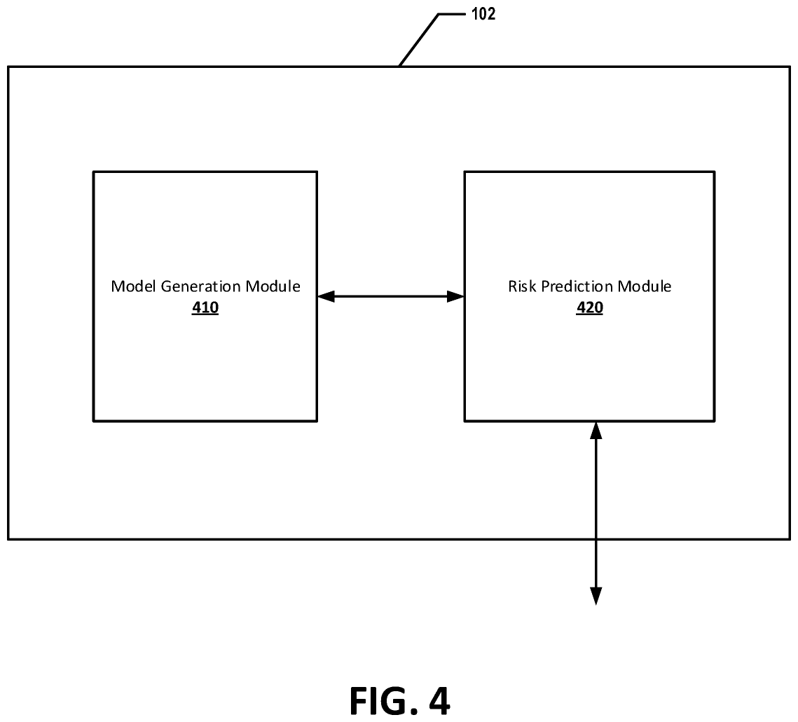

FIG. 4 provides a block diagram of an example system computing entity 102. In various embodiments, the system computing entity 102 comprises a model generation module 410. The model generation module 410 may be configured to generate a cohort predictive model based at least in part on historical data objects for a cohort of individuals that underwent a similar surgical operation or procedure (e.g., a surgical type cohort). In various embodiments, the model generation module 410 may also be configured to initialize or train a cohort predictive model. Every surgical procedure consists of physical intervention on a particular body system. Hence, the type of procedure specifies the organ, organ system, or tissue involved, as well as the degree of invasiveness. The influence of the type of surgery on development of chronic or persistent POP is well understood by those of skill in the art. Longer and more complicated operations are often linked with higher risks of chronic pain development, although the pattern is irregular and also tied to the type of tissue involved in the surgery. Thus, it may be appreciated that predictive models may be unique to surgery cohorts, and as such, system computing entity 102 (e.g., model generation module 410) may be configured to generate cohort-specific predictive models, or cohort predictive models. Specifically, model generation module 410 may be configured to generate cohort predictive models based at least in part on historical data objects each comprising multivariate intra-operative vital sign data and associated with a binary classification of mild or severe persistent POP.

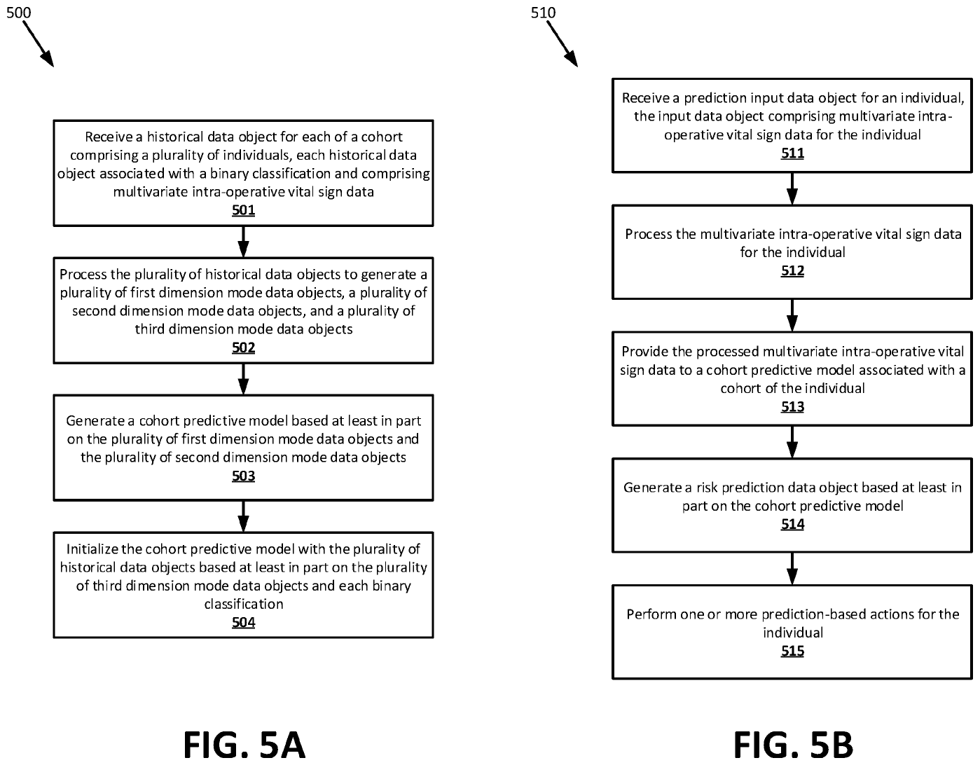

Accordingly, FIG. 5A provides a process 500 for generating and initializing a cohort predictive model. In various embodiments, operations of process 500 may be performed by the system computing entity 102 and/or the model generation module 410, and the system computing entity 102 may comprise means, such as processing element 205, memories 210, 215, network interface 220, and/or the like, for performing the operations of process 500.

As illustrated in FIG. 5A, process 500 comprises operation 501. In various embodiments, the process 500 begins with operation 501. Operation 501 comprises receiving a historical data object for each of a cohort comprising a plurality of individuals. Each historical data object is associated with a binary classification and comprises multivariate intra-operative vital sign data for an individual of the cohort. In various embodiments, the binary classification is a classification of whether the corresponding individual of the cohort experienced mild or severe persistent POP. The binary classification may correspond to a specific post-operative time period, timeframe, timepoint, and/or the like. For example, a binary classification may be a classification of whether the corresponding individual experienced mild or severe persistent POP at 30 days after a surgical operation, while another binary classification may be for 90 days after a surgical operation. In various embodiments, each historical data object may be associated with one or more binary classifications each corresponding to a different post-operative time period, timeframe, timepoint, and/or the like.

In various embodiments, the multivariate intra-operative vital sign data includes data collected for various different vital sign variate types (e.g., heart rate, respiratory tidal volume, blood pressure, blood oxygen, and/or the like) throughout an intra-operative time period. In various embodiments, the multivariate intra-operative vital sign data comprises hemodynamic data. The dynamic interaction between surgical perturbations to circulatory function, and the sympathetic/parasympathetic responses under general anesthesia to compensate them are reflected in variations in hemodynamic parameters during surgery. As the autonomic nervous system drives the function of the heart by increasing or decreasing heart rate, heart rate can therefore be used to characterize the autonomic nervous system. Arterial blood pressure may be used as an imperfect estimate of adequacy of tissue perfusion. Peripheral capillary oxygen saturation (SpO2), which measures the amount of oxygen in the blood, also contains relevant information on the state of the circulation, and consequently the autonomic state (the state of autonomic nervous system), during surgery. Breathing causes slow periodic variations in the baseline heart rate and also affects blood pressure. Hence, breath-related parameters like respiratory tidal volume and end-tidal CO2 provide additional information on the autonomic state, which provides insight to a risk of persistent POP.

Breathing is coupled with heart-rate variations through a centrally mediated mechanism, while it also mechanically perturbs aortic pressure, venous return, and pulmonary vascular. The cyclic variation in blood pressure resulting from breathing affects heart rate through autonomically mediated baroreceptor reflex. Fluctuations in peripheral vascular resistance is another source of perturbation to cardiovascular homeostasis, as vascular beds adjust local blood flow to balance demand and supply. These fluctuations perturb blood pressure and result in compensatory variations in heart rate.

The frequency content of variations in hemodynamic parameters that indirectly reflect the frequency bands for sympathetic and parasympathetic activities that compensate for short-term variations in heart rate and other hemodynamic parameters are concentrated in three fundamental spectral peaks: low-frequency peak, midfrequency peak, and high-frequency peak. The high-frequency peak (from 0.3 to 0.5 Hz) represents respiratory frequency and shifts with variations in respiratory rate. The midfrequency peak (from 0.09 to 0.15 Hz) describes blood pressure oscillations happening at lower frequency than respiratory frequency and is linked to the frequency response of the baroreceptor reflex. The low-frequency peak (from 0.02 to 0.09 Hz) is associated with fluctuations in vasomotor tone.

The discussed spectral characteristic of fluctuations in hemodynamic parameters is mainly associated with the activity of the sympathetic and parasympathetic nervous systems and the renin-angiotensin system to control cardiovascular responses, and specifically, the hemodynamic fluctuations happening at high frequencies (above approximately 0.1 Hz) are associated with the activity of the parasympathetic system. Meanwhile, hemodynamic fluctuations at lower frequencies may reflect the joint activity of sympathetic and/or parasympathetic nervous systems. The renin-angiotensin system is a hormonal system that regulates blood pressure and fluid and electrolyte balance, as well as systemic vascular resistance. Blockade of this system has been shown to drastically increase the amplitude of the lower frequencies.

Thus, to provide a comprehensive view of the state of the autonomic nervous system during surgery, the multivariate intra-operative vital sign data comprises hemodynamic parameter data collected with a high sampling rate. For example, in some embodiments, the multivariate intra-operative vital sign data is collected at a rate of one sample per second. The multivariate intra-operative vital sign data for the individual may comprise periodic measurements for heart rate, blood oxygen level, end-tidal CO2, respiratory tidal volume, systolic blood pressure, diastolic blood pressure, isoflurane concentration, sevoflurane concentration, and/or the like. In some other embodiments, the multivariate intra-operative vital sign data is collected at a rate of one sample per minute. This sampling rate restricts the analysis to a narrow band in lower frequencies. Since the observable changes in hemodynamic parameters and corresponding surgical decisions occur at intervals not shorter than one minute, this narrow band in lower frequencies remains informative for developing the cohort predictive model.

Process 500 further comprises operation 502. In various embodiments, operation 502 may follow operation 501. Operation 502 comprises processing the plurality of data objects to generate a plurality of first dimension mode data objects, a plurality of second dimension mode data objects, and a plurality of third dimension mode data objects.

In various embodiments, the plurality of historical data objects may be, may comprise, may be aggregated into, and/or the like, a third-order tensor. For example, the plurality of data objects may comprise a recording of I₁ intra-operative vital sign variate types (e.g., heart rate, respiratory tidal volume) over I₃ different patients in a surgical type cohort, with the intra-operative vital signs being recorded at I₂ time points for each patient. Intra-operative vital sign recordings that span different numbers of time points may be cut to a common window of time (e.g., I₂ time points) to fit in with this constraint. Thus, the multivariate intra-operative vital sign data for a plurality of patients may be represented as an I₁×I₂×I₃ array of vital signs, a third-order tensor such as Aϵ [symbol] ^{I}^{₁}^{×I}^{₂}^{×I}^{₃}. Each member of this tensor, aᵢ_{₁}ᵢ_{₂}ᵢ_{₃}, denotes the recorded value of vital-sign i₁ at time point i₂ for patient i₃.

In various embodiments, processing the plurality of data objects comprises processing multivariate intra-operative vital sign data for each patient individually. For example, a matrix A_{I}_{_{1X}}_{I}_{₂}, which holds the values for each vital sign i₁ and time point i₂ for one patient, may be obtained and then processed. In such embodiments, processing the matrix A_{I}_{_{1X}}_{I}_{₂} comprising multivariate intra-operative vital sign data for a patient comprises performing singular value decomposition (SVD) techniques on the matrix A_{I}_{_{1X}}_{I}_{₂}.

Equation 1 provides a SVD of the matrix A_{I}_{_{1X}}_{I}_{₂} into R number of components to approximate the original data matrix.

$$A = {\sum }_{r = 1}^{R} {σ}_{r} {A}_{r} ; {A}_{r} = {U}_{r} ∘ {V}_{r} . \qquad ( 1 )$$

In Equation 1, ○ denotes the outer product of the vectors. This decomposition provides a low-dimensional subspace (a new coordinate system) with R components to describe the original high-dimensional data with I₁ or I₂ original dimensions. Each component, indexed by r, holds a coefficient across vital signs, uᵣᵢ_{₁}, and a coefficient across points in time uᵣᵢ_{₂}. These coefficients can be accumulated into first dimension mode data objects Uᵣ with length I₁ and second dimension mode data objects Vᵣ with length I₂. These dimension mode data objects represent the multivariate-temporal dynamics discovered within the original data matrix. It may be appreciated that the first dimension mode data objects relate to the multivariate vital signs (e.g., multivariate mode data objects) while the second dimension mode data objects relate to the intra-operative timepoint (e.g., temporal mode data objects). Each coefficient or element of the multivariate and temporal mode data objects contains two important pieces of information. The absolute value of the coefficient provides a measure of the particular vital sign's (or intraoperative timepoint's) contribution for that mode. If the coefficient is complex valued, the angle defined by the real and imaginary parts provides an explanation of the phase of that coefficient or element in relation to the other coefficients or elements vibrating at the frequency associated with that particular mode.

In various embodiments, multivariate intra-operative vital sign data in a matrix A_{I}_{₁}_{I}_{₂} for each of the cohort of patients may be concatenated into an I₁×I₂I₃ matrix, whereupon SVD techniques are performed on the larger matrix. First dimension mode data objects and second dimension mode data objects are then generated, and the second dimension mode data objects (e.g., temporal mode data objects) may have length I₂I₃. However, the second dimension mode data objects may not capture common temporal dynamics across patients.

To capture common temporal dynamics across patients, higher-order singular value decomposition (HOSVD) techniques may be performed directly on the original data tensor Aϵ [symbol] ^{I}^{₁}^{×I}^{₂}^{×I}^{₃}, in various embodiments. Equation 2 provides a HOSVD of such a data tensor comprising the multivariate intra-operative vital sign data for all of the cohort of patients.

$$A = {\sum }_{{i}_{1}} {\sum }_{{i}_{2}} {\sum }_{{i}_{3}} {S}_{{i}_{1} {i}_{2} {i}_{3}} {U}_{{i}_{1}}^{( 1 )} ∘ {U}_{{i}_{2}}^{( 2 )} ∘ {U}_{{i}_{3}}^{( 3 )} . \qquad ( 2 )$$

In analogy to SVD, a first dimension mode data object U⁽¹⁾ may be a prototypical pattern across intra-operative vital sign variate types (e.g., a multivariate mode data object), and U⁽²⁾ may be a temporal dynamic across intra-operative timepoints (e.g., a temporal mode data object). These multivariate mode data objects and temporal mode data objects represent dynamics that are common among all patients in the cohort. A third dimension mode data object U⁽³⁾ may then represent patient-specific variations, or patient factors, for the multivariate-temporal dynamics.

To capture propagating dynamics, the multivariate intra-operative vital sign data—which are real-valued—are augmented with their Hilbert transforms to form a complex-valued third-order tensor such as X∈ [symbol] ^{I}^{₁}^{×I}^{₂}^{×I}^{₃}. In various embodiments, complex data may be obtained using any other technique. In some embodiments, the HOSVD techniques may then be performed on the complex-valued tensor X, and Equation 2 remains accurate in describing first dimension mode data objects, second dimension mode data objects, and third dimension mode data objects generated as a result of performing HOSVD techniques on the complex-valued third-order tensor X, which is also referred to herein as a complex HOSVD technique or a complex HOSVD. The complex HOSVD identifies dynamic factors that carry additional information related to phase. As a result, each coefficient or element of a first dimension mode data object, for example, may comprise and/or be associated with a magnitude and a phase. In various embodiments, the coefficients or elements of a first dimension mode data object have the same phase with the exception of the coefficient or element associated with the contribution of the tidal volume vital sign type. Thus, phase information for the plurality of historical data objects may be determined based at least in part on processing the plurality of historical data objects using complex HOSVD techniques.

In various embodiments, the first dimension mode data objects, the second dimension mode data objects, and the third dimension mode data objects may be significantly different across cohorts and correlate within cohorts. For example, the evolutionary dynamics of multivariate intra-operative vital sign data have at least one temporal mode significantly different across cohorts, or more specifically, performing complex HOSVD techniques on multivariate intra-operative vital sign data may result in at least one second dimension mode data object being significantly different between different cohorts.

In some embodiments, operation 502 comprises creating correntropy matrices based at least in part on the complex-valued third-order tensor X and performing the complex HOSVD techniques on the correntropy matrices, which is referred to herein as a robust complex HOSVD technique or a robust complex HOSVD.

In some embodiments, creating the correntropy matrices may comprise unfolding the complex-valued third-order tensor X∈ [symbol] ^{I}^{₁}^{×I}^{₂}^{×I}^{₃} to an (I₁×I₂I₃)—matrix X₍₁₎, an (I₂×I₃I₁)—matrix X₍₂₎, and an (I₃×I₁I₂)—matrix X₍₃₎, creating moment matrices based at least in part on the matrices X₍₁₎, X₍₂₎, and/or X₍₃₎, and creating the correntropy matrices based at least in part on one or more of the moment matrices. In some embodiments, creating the correntropy matrices comprises applying a cross-correntropy function to the random processes included in one or more of the moment matrices. By applying the cross-correntropy function, the complex values of hemodynamic responses associated with each time and vital sign can be implicitly mapped to a reproducing kernel Hilbert space (RKHS). The RKHS may be defined by the statistics of the random processes associated with different mode matrix unfoldings of X.

The cross-correntropy function for two stochastic processes {xₜ, t∈T} and {yₜ, t∈T} can be defined as in Equation 3.

V(t₁,t₂)=E[k(xₜ_{₁},yₜ_{₂})] (3)

In Equation 3, E[⋅] indicates mathematical expectation over the stochastic processes xₜ and yₜ. k(⋅,⋅) is a positive-definite kernel function that respects Mercer's conditions. By using a kernel function in the argument of the expectation operator, the kernel space induced by the correntropy includes statistical information of the data mapped into the new RKHS. By this means, the inner product in the new RKHS is responsive to overall data statistics, similar to the Mahalanobis distance, which defines a metric that depends on data statistics in the space spanned by data, except that here the Mercer kernel space is used instead. In correntropy, the data statistics enter in the definition of the inner product. By selecting a symmetric positive definite kernel function, Equation 3 becomes a symmetric and positive-definite function and gives a translation invariant similarity measure. Additionally, according to the Moore-Aronszajn theorem, there is a unique RKHS associated with the correntropy function. Given that a conventional correlation function is not necessarily positive definite, there exists no such RKHS associated with correlation function. Therefore, a substantial benefit of cross-correntropy function is that the cross-correntropy function uses the structure of a unique RKHS in the definition of the similarity measure. One common kernel function used in correntropy function is the Gaussian kernel given by Equation 4.

$${G}_{σ} ( {x}_{{t}_{1}} - {y}_{{t}_{2}} ) = \frac{1}{\sqrt{2 π} σ} {e}^{- \frac{{( {x}_{{t}_{1}} - {y}_{{t}_{2}} )}^{2}}{2 {σ}^{2}}} . \qquad ( 4 )$$

In Equation 4, σ denotes the variance of the data, called kernel width parameter or kernel size. The kernel width controls the impact of the higher-order moments in the similarity evaluation in Equation 3. By increasing the kernel width σ, the higher-order moments decay rapidly and eventually the second-order moment becomes dominant. Then cross-correntropy function reduces to the conventional correlation. In contrast, when σ is too small, a data point is only similar to itself. In this respect, the kernel function approximates the Dirac delta function, and cross-correntropy function will no longer characterize statistics of data. With an appropriate kernel width, cross-correntropy function weights higher-order moments to estimate any of the lₚ-norms.

The cross-correntropy function shares with the cross-correlation function the fact that it quantifies similarities among pairs of lags in time series. Given that the time varying contents of intra-operative vital signs represent a multivariate stochastic process, a robust complex HOSVD technique can be built based on the cross-correntropy function.

In some embodiments, the moment matrix H⁽¹⁾ is created based at least in part on the (I₁×I₂I₃)—matrix X₍₁₎, the first mode matrix unfolding of X. The cross-correntropy function is then applied to the random processes included in the moment matrix H⁽¹⁾ to generate a (I₁×I₁) correntropy matrix V⁽¹⁾, which is defined in Equation 5. Similarly, a (I₂×I₂) correntropy matrix V⁽²⁾ and a (I₃×I₃) correntropy matrix V⁽³⁾ can be generated by applying a cross-correntropy function to the random processes included in the moment matrix H⁽²⁾ and H⁽³⁾, respectively, wherein the moment matrix H⁽²⁾ and H⁽³⁾ are created based at least in part on the second mode matrix unfolding of X and the third mode matrix unfolding of X, respectively.

V⁽¹⁾=[V_{ii*}⁽¹⁾]=[V⁽¹⁾(Xᵢ_{₁}₌ᵢ,Xᵢ_{₁}_{=i*})]E[k(Xᵢ_{₁}₌ᵢ,Xᵢ_{₁}_{=i*})] (5)

In some embodiments, performing the complex HOSVD techniques on the correntropy matrices comprises the eigen-decomposition of the correntropy matrix. The correntropy matrix is analogous to the covariance matrix in the RKHS. Therefore, based on spectral theory, there exists a set of orthonormal bases and a set of positive real eigen values, such that the correntropy matrix is diagonal in this set of bases. In some embodiments, eigen directions may be extracted through singular value decomposition of the correntropy matrix.

To apply the SVD procedure to the correntropy matrix, the data should be zero mean in the feature space. In some embodiments, the data can be centered by subtracting the cross-information potential from the entries of correntropy matrix. In some other embodiments, a widely used approach in kernel methods to remove the mean value from the entries of the Gram Matrix can be employed and modified for centering the correntropy matrix. For example, let 1_{I}_{₁}_{×I}_{₁} indicate a (I₁×I₁) matrix with all entries equal to 1. The centered version of the correntropy matrix can be formulated as in Equation 6.

V^{(1)c}=V⁽¹⁾−1_{I}_{₁}_{×I}_{_{1·V/I}}₁−V⁽¹⁾·1_{I}_{₁}_{×I}_{₁}/I₁+1_{I}_{₁}_{×I}_{₁}·V⁽¹⁾·1_{I}_{₁}_{×I}_{₁}/I₁². (6)

In some embodiments, the eigenvectors can be obtained by singular value decomposition of the centered version of the correntropy matrix. For example, a first set of eigenvectors {eₖ⁽¹⁾}ₖ₌₁^{I}^{₁} can be obtained by singular value decomposition of V^{(1)c}. Similarly, a second set of eigenvectors {eₖ⁽²⁾}ₖ₌₁^{I}^{₂} and a third set of eigenvectors {eₖ⁽³⁾}ₖ₌₁^{I}^{₃} can be obtained by singular value decomposition of centered versions of the correntropy matrices V^{(2)c} and V^{(3)c}, respectively. The extracted singular vectors may provide the significant multivariate temporal descriptors available in the total intra-operative vital sign space, and can be used to form a multidimensional filter and to project the intra-operative vital signs into the subspace in some embodiments.

In some embodiments, the first set of eigenvectors can be accumulated into the first dimension mode data objects, the second set of eigenvectors can be accumulated into the second dimension mode data objects, and the third set of eigenvectors can be accumulated into the third dimension mode data objects.

Process 500 further comprises operation 503. In various embodiments, operation 503 may follow operation 502. Operation 503 comprises generating a cohort predictive model based at least in part on the plurality of first dimension mode data objects (e.g., multivariate mode data objects) and the plurality of second dimension mode data objects (e.g., temporal mode data objects).

In some embodiments, the first dimension mode data objects and the second dimension mode data objects extracted through applying complex HOSVD on the complex-valued tensor X are used to describe the physiological dynamic correlations and to provide insight into any lead-lag relations among individual responses expressed in instantaneous phases of the complex vital signs in a cohort predictive model. For example, the first dimension mode data objects and the second dimension mode data objects may be combined (e.g., by outer product) to form various components, as previously described. To obtain the most salient multivariate and temporal factors for generating a cohort predictive model, a rank feature method based at least in part on Fisher ranking techniques may be used to select a number of top ranked components. In various embodiments, the top three ranked components are selected. Using the selected components, a cohort predictive model is then generated. In various embodiments, the cohort predictive model comprises an n-dimensional data manifold or structure, where n corresponds to the number of selected components. For example, the cohort predictive model comprises a three-dimensional data manifold, where the three dimensions of the data manifold are based at least in part on three selected components. It may be appreciated that each cohort predictive model may be based at least in part on different dimensions. For example, a cohort predictive model for an orthopedic surgery cohort may have a dimension that strongly weighs the activation of blood oxygen levels late in the intra-operative time period, another dimension that strongly weighs the activation of respiratory tidal volume, and another dimension that strongly weighs the activation of a combination of heart rate and blood pressure both early and late in the intra-operative time period. Meanwhile, a cohort predictive model for a thoracic surgery cohort may have a dimension strongly weighing the heart rate and a separate dimension strongly weighing blood pressure.

In some other embodiments, the first dimension mode data objects and the second dimension mode data objects extracted through applying complex HOSVD on the correntropy matrices are used to describe the physiological dynamic correlations and to provide insight into how dynamics of intra-operative vital signs are associated with long-term post-operative pain development using a cohort predictive model. In some embodiments, to obtain the most salient multivariate and temporal factors for generating a cohort predictive model, a rank feature method based at least in part on Fisher ranking techniques may be used to select a number of top ranked components from the extracted first dimension mode data objects and/or second dimension mode data objects. In some embodiments, the top three ranked components providing the highest Fisher scores are selected to form a 3-dimensional data manifold. Using the selected components, a cohort predictive model is then generated. In various embodiments, the cohort predictive model comprises an n-dimensional data manifold or structure, where n corresponds to the number of selected components. For example, the cohort predictive model comprises a three-dimensional data manifold, where the three dimensions of the data manifold are based at least in part on the three selected components. It may be appreciated that each cohort predictive model may be based at least in part on different dimensions.

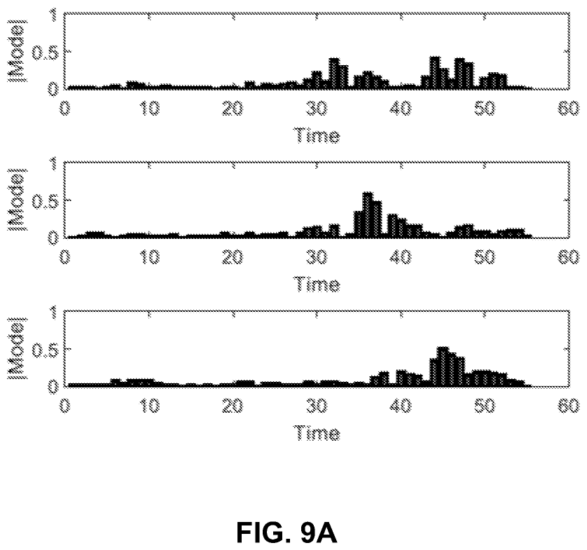

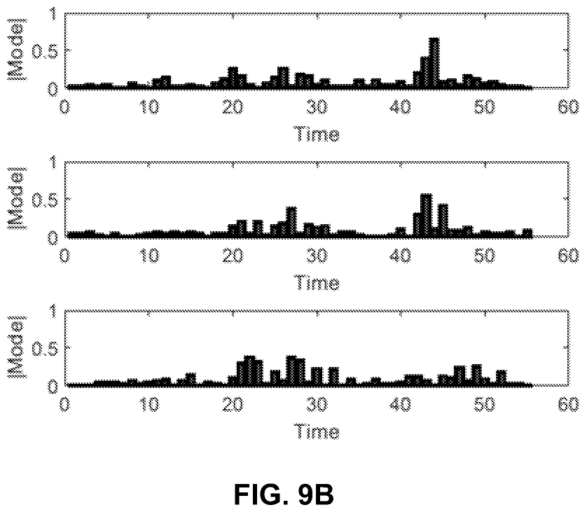

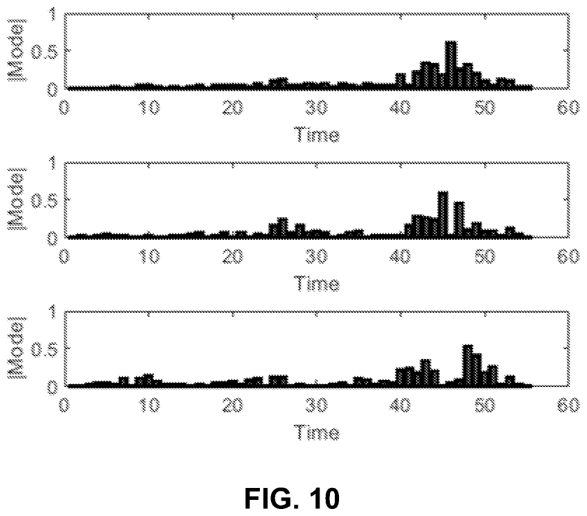

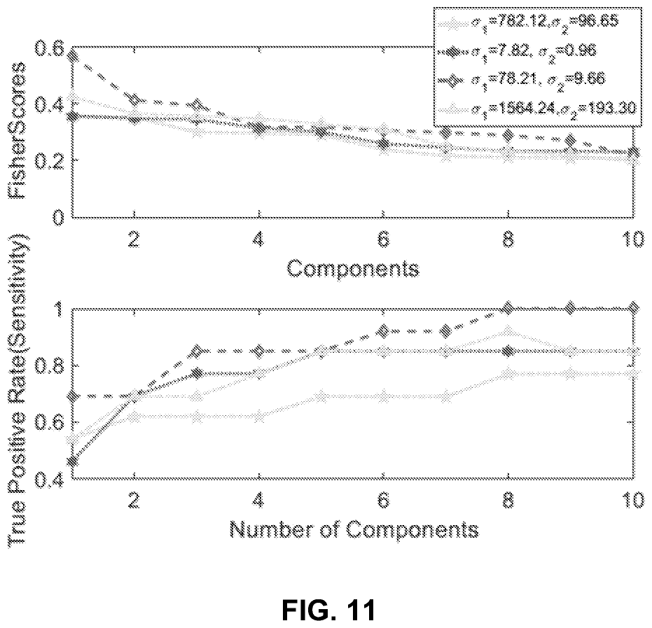

As aforementioned, creating the correntropy matrices comprises applying a cross-correntropy function to the random processes included in one or more of the moment matrices, and the kernel width controls the impact of the higher-order moments in the similarity evaluation. Some embodiments demonstrate a relation between the sparsity of temporal factors and the value of Fisher scores obtained for the most salient eigendirections (or the most salient components from the extracted first dimension mode data objects and/or second dimension mode data objects). For example, Fisher scores decrease for very small and very large kernel widths, as shown in FIG. 11, which displays how the value of Fisher scores changes in the top ten extracted components for different sets of Kernel width. FIGS. 9A and 9B show the first three temporal factors obtained using two different sets of kernel width σ₁=7.82, σ₂=0.96 and σ₁=782.12, σ₂=96.65, respectively, where σ₁ and σ₂ are kernel width parameters associated with moment matrices V^{(1)c} and V^{(2)c}. FIG. 10 shows the same temporal factors obtained using an optimal kernel width σ₁=78.21, σ₂=9.66. The temporal factors achieved using kernel width σ₁=78.21, σ₂=9.66 are sparser than those obtained by using kernel width σ₁=7.82, σ₂=0.96, and are denser than those obtained using kernel width σ₁=782.12, σ₂=96.65. In addition, as shown in FIG. 11, for very small and very large kernel widths, Fisher cores are spread over different components, which is not desirable. While for the optimal set of kernel width, the top three components contain the highest Fisher scores and hence show superior performance to model dissimilarity among categories of data.

FIG. 5A further illustrates process 500 comprising operation 504. In various embodiments, operation 504 may follow operation 503. Operation 504 comprises initializing the cohort predictive model with the plurality of historical data objects based at least in part on the plurality of third dimension mode data objects and each binary classification. As aforementioned, each historical data object may be associated with a binary classification. The binary classification of a historical data object may be determined based at least in part on a corresponding individual of the cohort reporting an average pain intensity on a numerical scale at a specific post-operative time period, timeframe, timepoint, and/or the like.

In some embodiments, initializing the cohort predictive model comprises projecting each historical data object onto the n-dimensional manifold of the cohort predictive model. As discussed earlier, each complex HOSVD component identifies sub-hemodynamic parameters (multivariate factor), with common intra-surgery temporal dynamics (temporal factor), which were deferentially activated across individuals of the cohort. Overall, the complex HOSVD model uncovers a reasonable portrait of surgical dynamics (population dynamics) in which distinct subsets of hemodynamic parameters are active at different times during surgery and whose variation across individuals of the cohort encoded individual dynamic variables.

In some embodiments, if the complex HOSVD technique is used in operation 502, initializing the cohort predictive model further comprises modifying phase information of each historical data object based at least in part on the plurality of third dimension mode data objects, or individual patient factors. In some embodiments, for a better representation of dynamics, it may be beneficial to associate each principal component (as one base of the subspace) to each dynamic mode of the individual's responses of the cohort individuals encoded in patient factors (e.g., the third dimension mode data objects). In some embodiment, the coordinate systems provided by the common multivariate-temporal factors and the multivariate-temporal dynamics of cohort individuals are not necessarily the same and are not aligned exactly. Given that all factors in complex HOSVD are complex-valued factors, the patient-specific variations for the identified multivariate-temporal dynamics contain scaling and rotational adjustments appearing in the outer product of the multivariate-temporal dynamics with the patient factors.

To compare the complex correlations between each hemodynamic response and the extracted multivariate-temporal dynamics, it is essential to have a common coordinate system for all individuals of the cohort. Simultaneously, to account for dynamic variation across patients, instead of rotating the dynamics, the complex conjugate of elements, given by the patient factors, may be used to scale and rotate the hemodynamic responses (e.g., the phase information) before projection onto the n-dimensional manifold of the cohort predictive model in some embodiments. The process can be done per complex HOSVD component separately. From a geometrical point of view, the process can be considered as an active transformation in which the position of a point changes in a coordinate system, as opposed to a passive transformation which changes the coordinate system in which the point is described.

Thus, in some embodiments, the phase information for each historical data object is modified, rotated, transformed, and/or the like based at least in part on the patient factors represented in the plurality of third dimension mode data objects, and subsequently projected onto the n-dimensional manifold (e.g., three-dimensional manifold) of the cohort predictive model.

In some embodiments, initialization of the cohort predictive model then comprises training the cohort predictive model with the binary classifications. For example, linear discriminant analysis (LDA) may be performed to discriminate between historical data objects with the binary classification of mild persistent POP and historical data objects with the binary classification of severe persistent POP within a three-dimensional manifold. As such, a relationship between phase information of the multivariate intra-operative vital sign data, which represents hemodynamic responses of individuals of the cohort, and mild or severe persistent POP may be determined.

As aforementioned, the binary classifications may be associated with a specific post-operative time period, timeframe, timepoint, and/or the like. For example, a first binary classification may be associated with mild or severe persistent POP at 30 days after surgical operation (e.g., post-operative), while a second binary classification may be associated with mild or severe persistent POP at 90 days after surgical operation (e.g., post-operative). As such, the cohort predictive model may be initialized to determine a relationship between phase information and mild or severe persistent POP at a specific post-operative time period, timeframe, timepoint, and/or the like. In various embodiments, one or more cohort predictive models may be generated for a cohort, each cohort predictive model associated with a specific post-operative time period, timeframe, timepoint, and/or the like. In various embodiments, one cohort predictive model may determine and store relationships between phase information and various post-operative time periods, timeframes, timepoints, and/or the like. Thus, through process 500, a cohort predictive model may leverage a linkage between the dynamics of individuals' responses to surgical stimulation and long-term post-operative pain development.

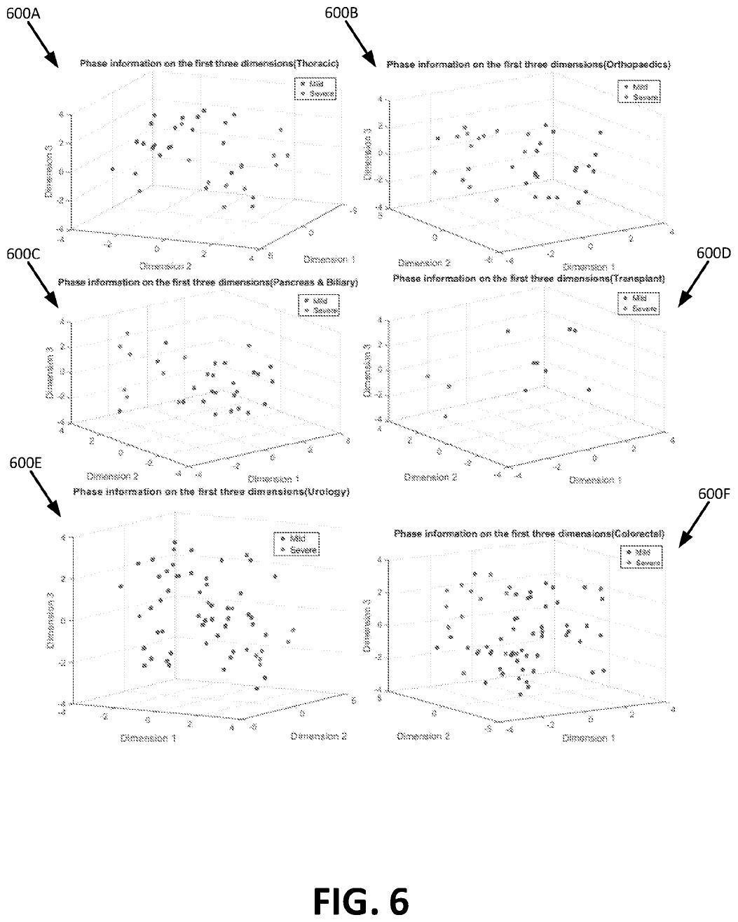

FIG. 6 illustrates portions of six example cohort predictive models with the complex HOSVD applied in operation 502. Specifically, FIG. 6 illustrates various three-dimensional manifolds 600 (e.g., 600A-F) each initialized with phase information of historical data objects for corresponding cohorts. The three-dimensional manifolds 600 are extracted by applying complex HOSVD on the complex-valued tensor X. As aforementioned, the cohorts may be surgical type cohorts. For example, three-dimensional manifold 600A corresponds to a thoracic surgery cohort, three-dimensional manifold 600B corresponds to an orthopedic surgery cohort, three-dimensional manifold 600C corresponds to a pancreatic/biliary surgery cohort, three-dimensional manifold 600D corresponds to a transplant surgery cohort, three-dimensional manifold 600E corresponds to a urology surgery cohort, and three-dimensional manifold 600F corresponds to a colorectal surgery cohort. It will be understood that in various embodiments, cohort predictive models may be generated and initialized for different surgical type cohorts, and may also be associated with other cohorts such as demographic cohorts.

After being projected onto each of the three-dimensional manifold 600, phase information of various historical data objects is shown in FIG. 6. Each historical data object is also associated with either mild or severe persistent POP. Thus, using discriminant analysis techniques such as LDA, a relationship or correlation may be determined between phase information and mild or severe persistent POP. For example, in three-dimensional manifold 600D for a transplant surgery cohort, historical data objects with a binary classification of severe persistent POP have phase information that is negative in the first dimension of the manifold, positive in the second dimension of the manifold, and negative in the third dimension of the manifold, whereas historical data objects with a binary classification of mild persistent POP have phase information projected as positive in the first dimension of the manifold.

As such, a relationship between phase information projected onto and/or in relation to dimensions of a three-dimensional manifold 600 and a binary classification of mild or severe POP may be determined. In some embodiments, each historical data object is associated with a non-binary classification indicating persistent POP. For example, the non-binary classification may be a numerical value within a range of persistent POP representative values. In such embodiments, a cohort predictive model may be initialized with multi-way discriminant analysis to determine relationships between phase information and each non-binary classification.

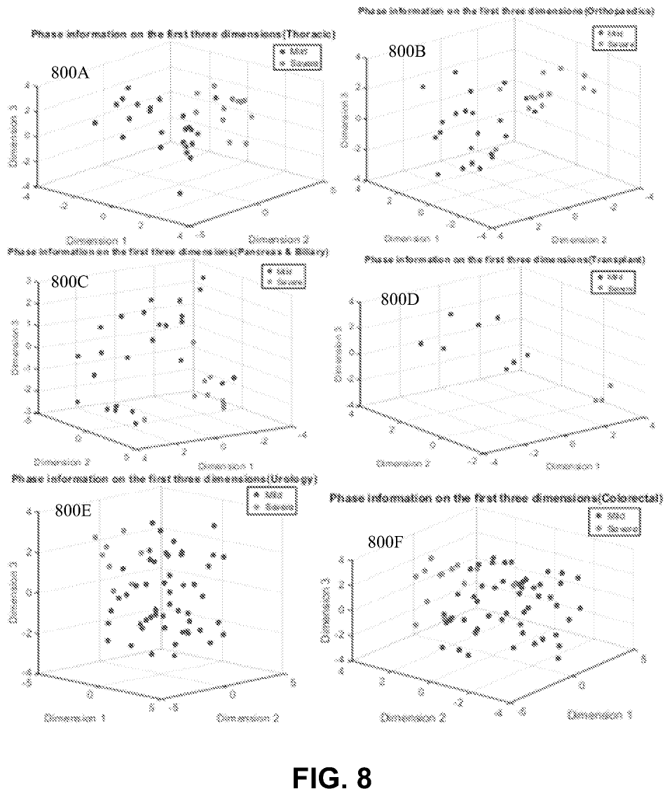

FIG. 8 illustrates portions of six example cohort predictive models with the robust complex HOSVD applied in operation 502. Specifically, FIG. 8 illustrates various three-dimensional manifolds 800 (e.g., 800A-F) each initialized with phase information of historical data objects for corresponding cohorts and the three-dimensional manifolds 800 are extracted by applying complex HOSVD on the correntropy matrixes generated from the complex-valued tensor X. As aforementioned, the cohorts may be surgical type cohorts. For example, three-dimensional manifold 800A corresponds to a thoracic surgery cohort, three-dimensional manifold 800B corresponds to an orthopedic surgery cohort, three-dimensional manifold 800C corresponds to a pancreatic/biliary surgery cohort, three-dimensional manifold 800D corresponds to a transplant surgery cohort, three-dimensional manifold 800E corresponds to a urology surgery cohort, and three-dimensional manifold 800F corresponds to a colorectal surgery cohort. As described above, it will be understood that in various embodiments, cohort predictive models may be generated and initialized for different surgical type cohorts, and may also be associated with other cohorts such as demographic cohorts. After being projected onto each of the three-dimensional manifold 800, phase information of various historical data objects is shown in FIG. 8. Each historical data object is also associated with either mild or severe persistent POP. Thus, using discriminant analysis techniques such as LDA, a relationship or correlation may be determined between phase information and mild or severe persistent POP. As such, a relationship between phase information projected onto and/or in relation to dimensions of a three-dimensional manifold 800 and a binary classification of mild or severe POP may be determined. In some embodiments, each historical data object is associated with a non-binary classification indicating persistent POP. For example, the non-binary classification may be a numerical value within a range of persistent POP representative values. In such embodiments, a cohort predictive model may be initialized with multi-way discriminant analysis to determine relationships between phase information and each non-binary classification.

### Prediction Module

Referring back to FIG. 4, system computing entity 102 may comprise a prediction module 420, in various embodiments. Prediction module 420 may be configured to generate a risk prediction data object for an individual of interest. The risk prediction data object generated by the prediction module 420 may be indicative at least a likelihood and/or a classification of whether the individual of interest will experience mild or severe persistent POP. As such, in various embodiments, the risk prediction data object comprises a binary classification of mild or severe persistent POP. In other embodiments, the risk prediction data object comprises a non-binary classification indicative of a degree of persistent POP. In various embodiments, the risk prediction data object is associated with a specific post-operative timeframe, timepoint, time period, and/or the like (e.g., 30 days post-operative, 90 days post-operative). In various embodiments, the risk prediction data object comprises a confidence score.

In various embodiments, the prediction module 420 may be configured to generate a risk prediction data object based at least in part on a cohort predictive model generated by a model generation module 410. For example, the prediction module 420 may communicate with the model generation module 410, such as to provide multivariate intra-operative vital sign data of an individual of interest and/or phase information of the multivariate intra-operative vital sign data, and to receive a classification (e.g., a binary classification of mild or severe persistent POP, a non-binary classification of a degree of persistent POP) from a cohort predictive model. In an example embodiment, the prediction module 420 may communicate with model generation module 410 via a model API.

Thus, system computing entity 102 (e.g., prediction module 420) is configured to perform operations for determining and predicting a risk of an individual to develop persistent POP, such as the operations provided in FIG. 5B. FIG. 5B illustrates an example process 510 for generating and determining a risk prediction data object for an individual indicative of a likelihood and/or classification of whether the individual will experience persistent POP. In various embodiments, system computing entity 102 comprises means, such as processing element 205, memories 210, 215, network interface 220, and/or the like, for performing each operation of process 510.

As illustrated in FIG. 5B, process 510 comprises operation 511. In various embodiments, process 510 may begin with operation 511. Operation 511 comprises receiving a prediction input data object for an individual, the prediction input data object comprising multivariate intra-operative vital sign data associated with the individual. For example, the prediction input data object may be received via network interface 220 from another computing entity. As another example, the prediction input data object may be received via a user interface. In various embodiments, the prediction input data object may be received via an API call or query.

As previously described, multivariate intra-operative vital sign data comprises data spanning a plurality of intra-operative timepoints for different vital sign variate types. For example, multivariate intra-operative vital sign data for the individual may include periodic measurements for heart rate, blood oxygen level, end-tidal CO2, respiratory tidal volume, systolic blood pressure, diastolic blood pressure, isoflurane concentration, sevoflurane concentration, and/or the like.

Process 510 further comprises operation 512. In various embodiments, operation 512 may follow operation 511. Operation 512 comprises processing the multivariate intra-operative vital sign data for the individual. In various embodiments, processing the prediction input data object comprises complexifying (e.g., by performing Hilbert transform techniques) the multivariate intra-operative vital sign data. Because only one individual is represented in the multivariate intra-operative vital sign data of the prediction input data object, higher-order techniques (e.g., complex HOSVD) are not necessary, as the individual or patient dimension is irrelevant. However, in various embodiments, the risk predictions of persistent POP for one or more individuals may be determined simultaneously by determining phase information using complex HOSVD.

Process 510 further comprises operation 513. In various embodiments, operation 513 may follow operation 512. Operation 513 comprises providing the processed (e.g., complexified) multivariate intra-operative vital sign data to a cohort predictive model associated with the cohort. In various embodiments, the processed (e.g., complexified) multivariate intra-operative vital sign data is provided to a cohort predictive model based at least in part on associating the prediction input data object with a cohort. In various embodiments, the cohort is a surgical type cohort. For example, the prediction input data object may be associated with one of (i) a thoracic surgery cohort, (ii) an orthopedic surgery cohort, (iii) a urological surgery cohort, (iv) a colorectal surgery cohort, (v) a transplant surgery cohort, and (vi) a pancreas/biliary surgery cohort.

In various embodiments, the prediction input data object may comprise additional data indicating a cohort with which the prediction input data object should be associated, and by extension which cohort predictive model to which the prediction input data object should be provided. For example, the prediction input data object may be associated with a specific surgical type cohort based at least in part on a medical record included in the prediction input data object and/or an indication to a specific surgical type. In various embodiments, the prediction input data object may be associated with a cohort based at least in part on analyzing the multivariate intra-operative vital sign data. It may be understood that various vital sign data patterns may exist specific to some surgical types, and thus, for example, a surgical type cohort may be determined based at least in part on the multivariate intra-operative vital sign data. In various embodiments, the prediction input data object may be associated with and/or classified as a specific surgical type cohort based at least in part on performing supervised machine learning methods.

Thus, the prediction input data object is provided to a cohort predictive model associated with a cohort associated with the prediction input data object, or a cohort to which the individual of interest belongs. In various embodiments, a cohort may be associated with one or more cohort predictive models each associated with a specific post-operative time period, timeframe, timepoint, and/or the like, and the prediction input data object is provided to each of the one or more cohort predictive models to generate one or more risk prediction data objects for different post-operative times. In other embodiments, a cohort may be associated with one cohort predictive model configured to provide classifications of persistent POP for different post-operative times, and the prediction input data object is provided to the cohort predictive model.

As illustrated in FIG. 5B, process 510 further comprises operation 514. In various embodiments, operation 514 may follow operation 513. Operation 514 comprises generating a risk prediction data object based at least in part on the cohort predictive model. In various embodiments, the cohort predictive model has been initialized, and a relationship between phase information and classifications (e.g., binary, non-binary) for persistent POP has been determined. Thus, based at least in part on the phase information of the processed (e.g., complexified) multivariate intra-operative vital sign data of the prediction data object, a classification for a predicted risk of persistent POP for the individual of interest may be determined and generated. In various embodiments, the risk prediction data object comprises the classification for a predicted risk of persistent POP for the individual.

Specifically, as previously described, the cohort predictive model may comprise a n-dimensional manifold, upon which the complexified multivariate intra-operative vital sign data of the individual of interest may be projected. In various embodiments, the cohort predictive model may be initialized with historical data objects (e.g., in operation 504) such that a classification for the individual of interest may be determined based at least in part on the projection of the complexified multivariate intra-operative vital sign data of the individual of interest. In some embodiments, a classification for the individual may be determined based at least in part on the phase information of the projection of the complexified multivariate intra-operative vital sign data onto the n-dimensional manifold. In some embodiments, an axis within the n-dimensional manifold (e.g., three-dimensional manifold 600) may be determined based at least in part on discriminant analysis (e.g., LDA), and a classification for the individual may be determined based at least in part on the phase information of the projection of the complexified multivariate intra-operative vital sign data of the individual of interest within the n-dimensional manifold onto the axis. In various embodiments, a binary classification of mild or severe persistent POP for the individual may be determined. In various embodiments, a non-binary classification of a degree of persistent POP may be determined.

Furthermore, a classification for a predicted risk of persistent POP for the individual of interest may be associated with a specific post-operative time period, timeframe, timepoint, and/or the like. For example, the cohort predictive model may determine a relationship between phase information and persistent POP for 30 days post-operative, and using the relationship, determine a classification for a predicted risk of persistent POP at 30 days post-operative for the individual of interest.

Thus, the cohort predictive model may provide a classification, and a risk prediction data object comprising the classification may be generated. In various embodiments, the risk prediction data object comprises one or more classifications each associated with a different post-operative time, and as such, the risk prediction data object provides a predicted risk across a post-operative time period. In various embodiments, the risk prediction data object comprises a confidence score in the classification or prediction. In various embodiments, the risk prediction data object comprises the selected n dimensions of the cohort predictive model.

As illustrated in FIG. 5B, process 510 further comprises operation 515. In various embodiments, operation 515 may follow operation 514. Operation 515 comprises performing one or more risk prediction-based actions for the individual. In various embodiments, the one or more risk prediction-based actions comprises displaying the risk prediction data object, the binary classification of whether the individual will develop persistent POP, and/or the binary classification of whether the individual will develop mild or severe persistent POP. In various embodiments, the first dimension mode data objects and the second dimension mode data objects may also be displayed. In various embodiments, the one or more risk prediction-based actions comprises transmitting the risk prediction data object to a client computing entity 106 associated with the individual. For example, the risk prediction data object may be provided in an API response in response to an API call.

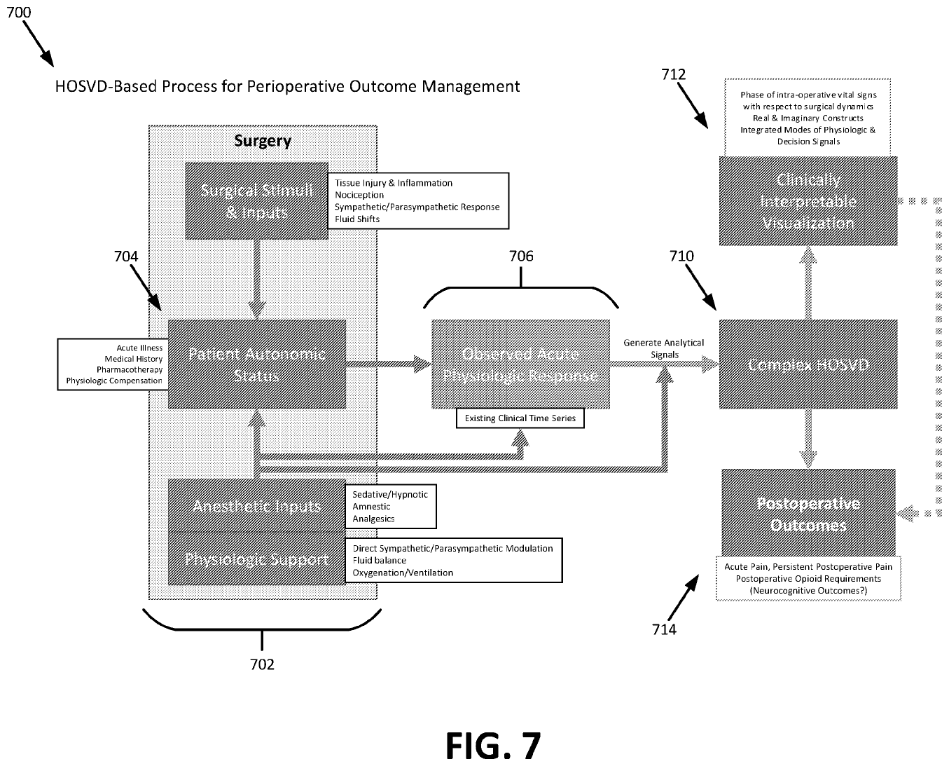

Referring now to FIG. 7, a diagram 700 for a general overview of predicting a risk of persistent POP for an individual of interest is provided. As illustrated in the diagram 700, various factors during a surgical operation 702 may affect the patient's autonomic status 704, which is manifested as multivariate intra-operative vital sign data 706. For example, surgical stimuli and inputs, anesthetic inputs, and physiologic supports may all impact the patient's autonomic status 704. The patient's autonomic status 704 is reflected in multivariate intra-operative vital sign data 706, or observed acute physiologic response to the surgical operation 702.

As illustrated, at operation 710, complex HOSVD techniques may be performed on the multivariate intra-operative vital sign data 706 to determine and extract phase information from the multivariate intra-operative vital sign data 706. Such phase information, along with additional data such as dimension mode data objects, may be visualized and/or displayed at operation 712. Meanwhile, phase information determined from performing complex HOSVD techniques at operation 710 may be used to determine and predict post-operative outcomes at operation 714. That is, a prediction of whether an individual may develop persistent POP and/or whether an individual may develop mild or severe persistent POP may be determined at operation 714 based at least in part on phase information determined from complex HOSVD techniques. Predictions of post-operative outcomes may be further processed or applied, such as to determine post-operative opioid requirements, or other medication requirements.

### V. Computer Program Products

Embodiments of the present disclosure may be implemented in various ways, including as computer program products that comprise articles of manufacture. Such computer program products may include one or more software components including, for example, software objects, methods, data structures, and/or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of a data structure, apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises a combination of computer program products and hardware performing certain steps or operations.

Embodiments of the present disclosure are described above with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### VI. Conclusion

It should be understood that the examples and embodiments described herein are for illustrative purposes only and that various modifications or changes in light thereof will be suggested to persons skilled in the art and are to be included within the spirit and purview of this application. Although the present disclosure is considered complete and comprehensive, additional context and insight may be gleaned from the appendices attached alongside this specification (which describes generally systems, apparatuses, and methods in accordance with embodiments herein). It should be understood that the examples and embodiments in Appendices A and B are also for illustrative purposes and are non-limiting in nature. The contents of Appendices A and B are incorporated herein by reference in their entirety.

Many modifications and other embodiments of the present disclosure set forth herein will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claim concepts. Although specific terms are employed herein, they are used in a generic and descriptive sense only and not for purposes of limitation.

1. A computer-implemented method for predicting a risk of persistent post-operative pain for an individual, the computer-implemented method comprising:
receiving, by one or more processors, a prediction input data object comprising multivariate intra-operative vital sign data of the individual;
processing, by the one or more processors, the multivariate intra-operative vital sign data of the individual;
providing, by the one or more processors, at least the processed multivariate intra-operative vital sign data to a cohort predictive model associated with a cohort of the individual, wherein the cohort predictive model is initialized with historical data objects associated with a post-operative timepoint;
generating, by the one or more processors, a risk prediction data object comprising a classification of phase information determined based at least in part on the cohort predictive model, wherein the risk prediction data object is associated with the post-operative timepoint; and
initiating, by the one or more processors, the performance one or more risk prediction-based actions for the individual.

2. The computer-implemented method of claim 1, wherein processing the multivariate intra-operative vital sign data comprises complexifying the multivariate intra-operative vital sign data of the individual, and wherein providing at least the processed multivariate intra-operative vital sign data to a cohort predictive model comprises projecting the processed multivariate intra-operative vital sign data onto a three-dimensional manifold of the cohort predictive model and determining phase information of the projection of the processed multivariate intra-operative vital sign data.

3. The computer-implemented method of claim 1, wherein the cohort predictive model is generated and initialized based at least in part by:
receiving a historical data object for each of a cohort comprising a plurality of individuals, each historical data object associated with a binary classification and comprising multivariate intra-operative vital sign data for a corresponding individual;
processing the plurality of historical data objects to generate a plurality of first dimension mode data objects, a plurality of second dimension mode data objects, and a plurality of third dimension mode data objects;
generating a cohort predictive model based at least in part on the plurality of first dimension mode data objects and the plurality of second dimension mode data objects, wherein the plurality of first dimension mode data objects and the plurality of second dimension mode data objects are processed to generate a three-dimensional manifold; and
initializing the cohort predictive model with the plurality of historical data objects based at least in part on the plurality of third dimension mode data objects and each binary classification.

4. The computer-implemented method of claim 3, wherein the plurality of historical data objects is aggregated and processed together using complex higher-order singular value decomposition (HOSVD), and wherein the three-dimensional manifold is generated based at least in part on ranks of components generated by the HOSVD.

5. The computer-implemented method of claim 3, wherein:
each of the plurality of first dimension mode data objects comprises a weight for each of one or more vital sign variate types;
each of the plurality of second dimension mode data objects comprises a weight for each of a plurality of intra-operative timepoints; and
each of the plurality of third dimension mode data objects comprises a weight for each of the plurality of individuals.

6. The computer-implemented method of claim 3, wherein initializing the cohort predictive model comprises determining a relationship between phase information of the projection of the plurality of historical data objects onto the three-dimensional manifold and a binary classification.

7. The computer-implemented method of claim 3, wherein:
the plurality of first dimension mode data objects comprises eigenvectors of a first correntropy matrix, wherein the first correntropy matrix is generated based at least in part on the plurality of historical data objects;
the plurality of second dimension mode data objects comprises eigenvectors of a second correntropy matrix, wherein the second correntropy matrix is generated based at least in part on the plurality of historical data objects; and
the plurality of third dimension mode data objects comprises eigenvectors of a third correntropy matrix, wherein the third correntropy matrix is generated based at least in part on the plurality of historical data objects.

8. The computer-implemented method of claim 7, wherein:
the first correntropy matrix is generated by applying a first cross-correntropy function to a first moment matrix, wherein the first moment matrix is generated based at least in part on a first mode matrix unfolding of a third-order tensor;
the second correntropy matrix is generated by applying a second cross-correntropy function to a second moment matrix, wherein the second moment matrix is generated based at least in part on a second mode matrix unfolding of the third-order tensor; and
the third correntropy matrix is generated by applying a third cross-correntropy function to a third moment matrix, wherein the third moment matrix is generated based at least in part on a third mode matrix unfolding of the third-order tensor, wherein the third-order tensor represents the plurality of historical data objects.

9. The computer-implemented method of claim 8, wherein each of the first, second, and third cross-correntropy functions is based on a Gaussian function.

10. The computer-implemented method of claim 1, wherein the one or more risk prediction-based actions for the individual comprises displaying the risk prediction data object with a three-dimensional manifold, wherein the three-dimensional manifold is generated based at least in part on the historical data objects.

11. An apparatus for predicting a risk of persistent post-operative pain for an individual, the apparatus comprising one or more processors and at least one non-transitory memory including program code, the at least one non-transitory memory and the program code configured to, with the one or more processors, cause the apparatus to at least:
receive a prediction input data object comprising multivariate intra-operative vital sign data of the individual;
process the multivariate intra-operative vital sign data of the individual;
provide at least the processed multivariate intra-operative vital sign data to a cohort predictive model associated with a cohort of the individual, wherein the cohort predictive model is initialized with historical data objects associated with a post-operative timepoint;
generate a risk prediction data object comprising a classification of phase information determined based at least in part on the cohort predictive model, wherein the risk prediction data object is associated with the post-operative timepoint; and
initiate the performance one or more risk prediction-based actions for the individual.

12. The apparatus of claim 11, wherein processing the multivariate intra-operative vital sign data comprises complexifying the multivariate intra-operative vital sign data of the individual, and wherein providing at least the processed multivariate intra-operative vital sign data to a cohort predictive model comprises projecting the processed multivariate intra-operative vital sign data onto a three-dimensional manifold of the cohort predictive model and determining phase information of the projection of the processed multivariate intra-operative vital sign data.

13. The apparatus of claim 11, wherein the cohort predictive model is generated and initialized based at least in part by:
receiving a historical data object for each of a cohort comprising a plurality of individuals, each historical data object associated with a binary classification, and comprising multivariate intra-operative vital sign data for a corresponding individual;
processing the plurality of historical data objects to generate a plurality of first dimension mode data objects, a plurality of second dimension mode data objects, and a plurality of third dimension mode data objects;
generating a cohort predictive model based at least in part on the plurality of first dimension mode data objects and the plurality of second dimension mode data objects, wherein the plurality of first dimension mode data objects and the plurality of second dimension mode data objects are processed to generate a three-dimensional manifold; and
initializing the cohort predictive model with the plurality of historical data objects based at least in part on the plurality of third dimension mode data objects and each binary classification.

14. The apparatus of claim 13, wherein the plurality of historical data objects is aggregated and processed together using complex higher-order singular value decomposition (HOSVD), and wherein the three-dimensional manifold is generated based at least in part on ranks of components generated by the HOSVD.

15. The apparatus of claim 13, wherein:
each of the plurality of first dimension mode data objects comprises a weight for each of one or more vital sign variate types;
each of the plurality of second dimension mode data objects comprises a weight for each of a plurality of intra-operative timepoints; and
each of the plurality of third dimension mode data objects comprises a weight for each of the plurality of individuals.

16. The apparatus of claim 13, wherein initializing the cohort predictive model comprises determining a relationship between phase information of the projection of the plurality of historical data objects onto the three-dimensional manifold and a binary classification.

17. The apparatus of claim 13, wherein:
the plurality of first dimension mode data objects comprises eigenvectors of a first correntropy matrix, wherein the first correntropy matrix is generated based at least in part on the plurality of historical data objects;
the plurality of second dimension mode data objects comprises eigenvectors of a second correntropy matrix, wherein the second correntropy matrix is generated based at least in part on the plurality of historical data objects; and
the plurality of third dimension mode data objects comprises eigenvectors of a third correntropy matrix, wherein the third correntropy matrix is generated based at least in part on the plurality of historical data objects.

18. The apparatus of claim 17, wherein:
the first correntropy matrix is generated by applying a first cross-correntropy function to a first moment matrix, wherein the first moment matrix is generated based at least in part on a first mode matrix unfolding of a third-order tensor;
the second correntropy matrix is generated by applying a second cross-correntropy function to a second moment matrix, wherein the second moment matrix is generated based at least in part on a second mode matrix unfolding of the third-order tensor; and
the third correntropy matrix is generated by applying a third cross-correntropy function to a third moment matrix, wherein the third moment matrix is generated based at least in part on a third mode matrix unfolding of the third-order tensor, wherein the third-order tensor represents the plurality of historical data objects.

19. The apparatus of claim 18, wherein each of the first, second, and third cross-correntropy functions is based on a Gaussian function.

20. The apparatus of claim 11, wherein the one or more risk prediction-based actions for the individual comprises displaying the risk prediction data object with a three-dimensional manifold, wherein the three-dimensional manifold is generated based at least in part on the historical data objects.

21. One or more non-transitory computer-readable storage media including instructions that, when executed by one or more processors, cause the one or more processors to:
receive a prediction input data object comprising multivariate intra-operative vital sign data of the individual;
process the multivariate intra-operative vital sign data of the individual;
provide at least the processed multivariate intra-operative vital sign data to a cohort predictive model associated with a cohort of the individual, wherein the cohort predictive model is initialized with historical data objects associated with a post-operative timepoint;
generate a risk prediction data object comprising a classification of phase information determined based at least in part on the cohort predictive model, wherein the risk prediction data object is associated with the post-operative timepoint; and
initiate the performance one or more risk prediction-based actions for the individual.
